(19) United States
(12) Patent Application Publication
(10) Pub. No.: US 2024/0416118 A1
(43) Pub. Date: Dec. 19, 2024

(54) CAPACITANCE BASED DC CHARGE-DISCHARGE CANCER TREATMENT DEVICE AND METHOD

(71) Applicant: REVOTERA MEDIKAL TEKNOLOJILER ANONIM SIRKETI, Kayseri (TR)

(72) Inventor: Purwo Taruno WARSITO, Tangerang (ID)

(73) Assignee: REVOTERA MEDIKAL TEKNOLOJILER ANONIM SIRKETI, Kayseri (TR)

(21) Appl. No.: 18/702,345

(22) PCT Filed: Aug. 15, 2022
(86) PCT No.: PCT/TR2022/050854

§ 371 Date: Apr. 18, 2024

(30) Foreign Application Priority Data
Oct. 18, 2021 (TR) 2021/016189

(51) Int. Cl. A61N 1/36; A61N 1/04

(52) U.S. Cl. CPC A61N 1/36002; A61N 1/0484; A61N 1/36034

(57) ABSTRACT

A cancer treatment device for preventing spreading of the cancer cells which exist at a target region is provided. The cancer treatment device comprises at least one first capacitive electrode for being positioned in the vicinity of the target region; a second capacitive electrode for being positioned against the first capacitive electrode such that the target region is positioned in between, and a signal generator for applying a wave-formed DC signal to the first capacitive electrode and to the second capacitive electrode in order to provide formation of a variable electrical field between the first capacitive electrode and the second capacitive electrode.

### CROSS REFERENCE TO THE RELATED APPLICATIONS

This application is the national phase entry of International Application No. PCT/TR2022/050854, filed on Aug. 15, 2022, which is based upon and claims priority to Turkish Patent Application No. TR2021/016189, filed on Oct. 18, 2021, the entire contents of which are incorporated herein by reference.

### TECHNICAL FIELD

The present invention relates to a cancer (including benign and malignant tumors) treatment device for treating of the cancer cells and preventing its recurrence and spread which exist in a target region of an individual.

### BACKGROUND

Cancer cells have a higher electrical (dielectric) structure when compared with normal cells, therefore, when cancer cells are subjected to external electrical field, they face stronger electrical polarization when compared with normal cells.

In cell division process, one characteristic of the cancer cells is that they have an uncontrollably high reproduction speed, and the electrical characteristics of the cells substantially increase as a result of cell bio-physical processes which are closely associated with the activity of the micro-tubules which exist in the cell nucleus. Micro-tubules play an important role in cell division process; first of all, in order to form two identical cell nuclei, micro-tubules attract replicated chromosomes, named as chromatid, towards the two division poles. Micro-tubules are macro-protein compounds formed by polymerization process of smaller compounds named as tubulin dimers which construct spiral tubes having a pore at the center thereof and similar to the fiber threads at the cell nucleus microscopically. Tubulin dimers which have high electrical polarity form the structure of micro-tubule polymer based on positive-negative electrostatic attraction named as van Der Waals bond. Van Der Waals bond is a weak electrical bond which is very sensitive against the effect of external electrical field. The presence of an external electrical field may affect the orientation of tubulin dimer and may prevent the polymerization process for forming micro-tubular fibers, and consequently the chromatid separation process can be intervened during the cell division process, thus, since the cell division process does not function well, leading to self-destruction.

In the present art, it is known that AC electrical field, generated by means of conductive electrodes attached to the skin, prevents growth of the cancer cells at the region which remains between the electrodes. In the application with number US2007225766, this technology is described. In such systems, conductive gel is used for contact with the body and this leads to subjecting of the patients to electrical current. Since the amount of current which can be applied without giving damage to human body is limited, efficiency of the treatment decreases. When the area of electrodes is shorter than the distance between the two electrodes, fringing effect occurs and the intensity and field vectors of these fringes cannot be accurately controlled. Additionally, the desired intensity may not be given in case the tumor is at various positions such as metastatic cases to different body organs.

As a result, because of the abovementioned problems, an improvement is required in the related technical field.

### SUMMARY

The present invention relates to a cancer treatment device, for eliminating the abovementioned disadvantages and for bringing new advantages to the related technical field.

An object of the present invention is to treat the cancer cells while preventing its recurrence and spread of the cancer cells without needing electrode contact to the skin directly.

Another object of the present invention is to provide movement freedom to the patient during treatment.

Another object of the present invention is to provide a system and method where the electrical field intensity can be adjusted within a wider range when compared with the present methods and where the deviations in the field are reduced.

In order to realize the abovementioned objects and the objects which are to be deducted from the detailed description below, the present invention is a cancer treatment device for treating cancer cells and preventing of its recurrence and spread which exist at a target region. Accordingly, said cancer treatment device comprises at least one first capacitive electrode for being positioned in the vicinity of said target region; a second capacitive electrode for being positioned against said first capacitive electrode such that the target region is positioned in between, and a signal generator for applying wave-formed DC signal to the first capacitive electrode and to the second capacitive electrode in order to provide formation of variable electrical field between said first capacitive electrode and said second capacitive electrode. Thus, the capacitive electrodes are periodically charged at opposite polarization and this leads to electrical polarization on the outer face of the target region. As a result of electrical polarization, electrical waves are generated and these electrical waves penetrate in mediums of dielectric materials including air, fat or other body tissues which are not necessarily electrically conductive. By means of this, electrical field with desired intensity can be applied. Additionally, reproduction of the cancer cells, which may exist in the body organs separated from the external surface of the body by body insulation layers where electrical current cannot pass, can also be prevented.

In a possible embodiment of the present invention, said first capacitive electrode and said second capacitive electrode are positioned such that a dielectrophoresis gap, which is equal to a predetermined distance, is provided between the outer face of the target region and the first capacitive electrode and the second capacitive electrode.

In another possible embodiment of the present invention, said distance is at least 1 mm. Thus, electrical field gradient occurs in the dielectrophoresis gap, and the dielectric constant difference between the capacitive electrodes and the target region surface increases the electrical field force (dielectrophoresis) applied to the target region.

In another possible embodiment of the present invention, said signal generator is configured to generate a signal which has square waveform.

In another possible embodiment of the present invention, the signal generator is configured to generate signal with 50% duty cycle.

In another possible embodiment of the present invention, the signal generator is configured to generate signal with frequencies which change between 100 kHz and 3 MHz.

In another possible embodiment of the present invention, said cancer treatment device comprises at least one grounding electrode associated with the grounding terminal and provided in the vicinity of the target region in a manner extending substantially parallel to the electrical field formed by the first capacitive electrode and the second capacitive electrode. Thus, electrical field is passed through the target region by minimizing fringing effect at the vicinity reducing the energy inefficiency while increasing treatment effectiveness.

In another possible embodiment of the present invention, two grounding electrodes are provided and are positioned mutually on two sides of the target region.

The present invention is moreover cancer treatment clothing for preventing spread of cancer cells which exist at a target region. Accordingly, the novelty is that the present invention comprises a cancer treatment device as mentioned in any one of the above.

In another possible embodiment of the present invention, said cancer treatment clothing is upper body clothing.

In another possible embodiment of the present invention, said cancer treatment clothing is lower body clothing.

In another possible embodiment of the present invention, said cancer treatment clothing is a headwear.

In another possible embodiment of the present invention, said cancer treatment clothing is a vest.

In another possible embodiment of the present invention, said cancer treatment clothing comprises a front wall, a rear wall positioned against said front wall, and side walls positioned mutually and which bind said rear wall and said front wall to each other; the first capacitive electrode is provided on said front wall, the second capacitive electrode is provided on said rear wall.

In another possible embodiment of the present invention, one each grounding electrodes are provided on said side walls.

In another possible embodiment of the present invention, said cancer treatment clothing is a short.

In another possible embodiment of the present invention, in order to define a dielectrophoresis gap, the cancer treatment clothing is provided in abundant dimension such that there is distance of at least 1 mm as from the body of the user.

The present invention is moreover cancer treatment bed for preventing spread of cancer cells which exist at a target region. Accordingly, the novelty is that the present invention comprises a cancer treatment device as mentioned in any one of the above.

In another possible embodiment of the present invention, the cancer treatment bed comprises at least one base electrode where the first capacitive electrode is connected, a bed layer for lying of the patient and for providing a dielectrophoresis gap between said base electrode and the patient, a coverlet for covering the patient; a middle part provided longitudinally in the middle of said coverlet is connected to the second capacitive electrode.

In another possible embodiment of the present invention, the grounding electrode is provided at the edge parts on the two edges of the middle part.

In another possible embodiment of the present invention, the cancer treatment bed comprises a base part which is hollow and having open upper wall, a bed layer provided such that a dielectrophoresis gap remains between the base of said base part and said bed layer, a cover which will cover the bed layer and which will cover the patient who lies on the bed layer.

The present invention is moreover a method applied by a treatment bed, treatment clothing or treatment device as mentioned in any one of the above for treating of the cancer cells and preventing its recurrence and spread which exist in a target region of an individual. Accordingly, the novelty is that the present invention is that a variable electrical field, which passes through the target region, is formed by a signal generator by means of at least one first capacitive electrode which is in the vicinity of said target region and having dielectrophoresis gap of at least 1 mm with respect to said target region, and by means of a second capacitive electrode which is against said first capacitive electrode and having dielectrophoresis gap of at least 1 mm with respect to said target region such that the target region is positioned between said first capacitive electrode and said second capacitive electrode.

### BRIEF DESCRIPTION OF THE DRAWINGS

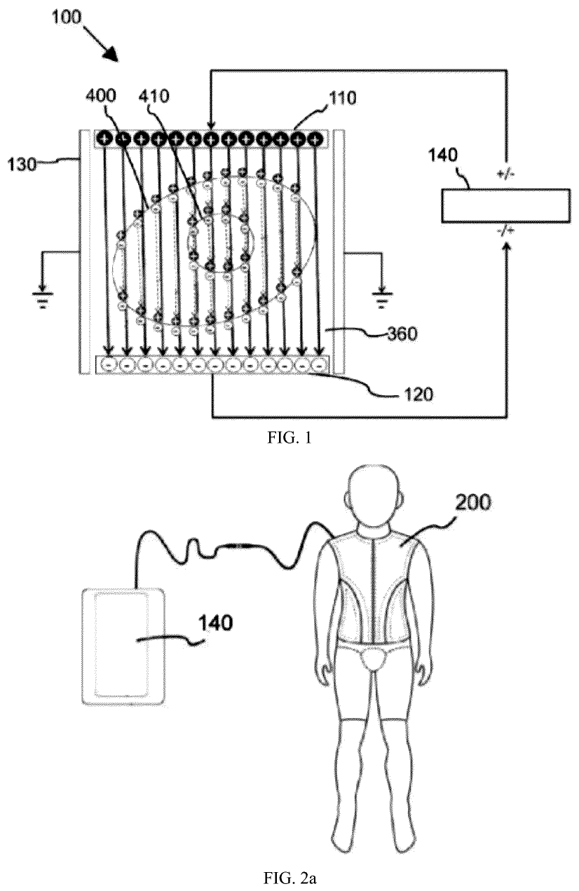

In FIG. 1, a representative view of an exemplary embodiment of the present invention is given.

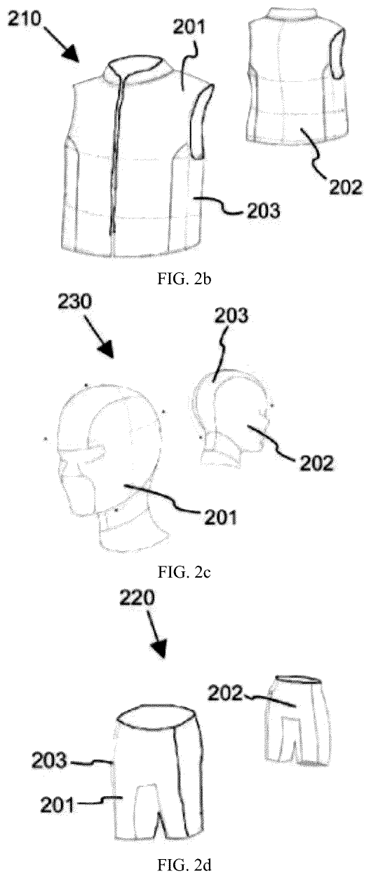

In FIG. 2a, 2b, 2c, 2d, representative views of the embodiment where the present invention is integrated to the clothing are given.

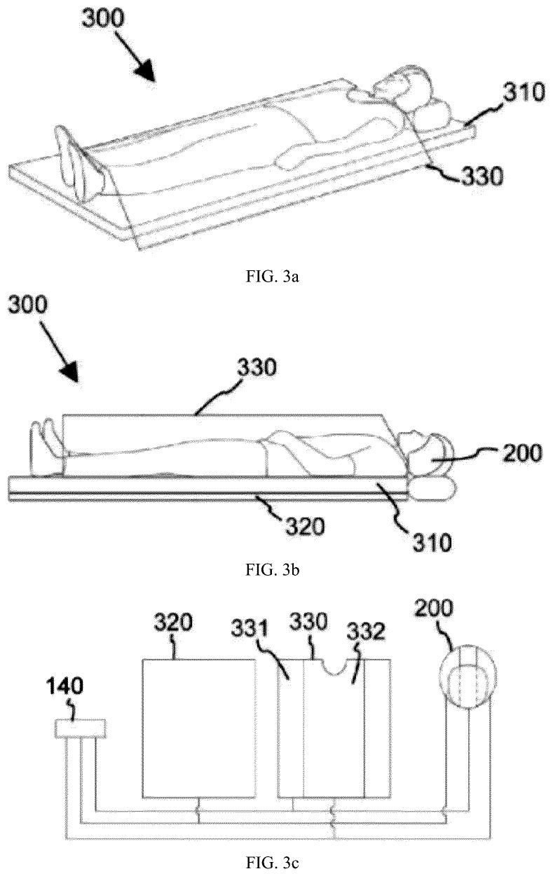

In FIG. 3a, 3b, representative views of the embodiment where the present invention is integrated to the bed is given.

In FIG. 3c, a representative view of the connections of an embodiment of the present invention, where the embodiment integrated to the bed and the embodiment integrated to the clothing are used together, is given.

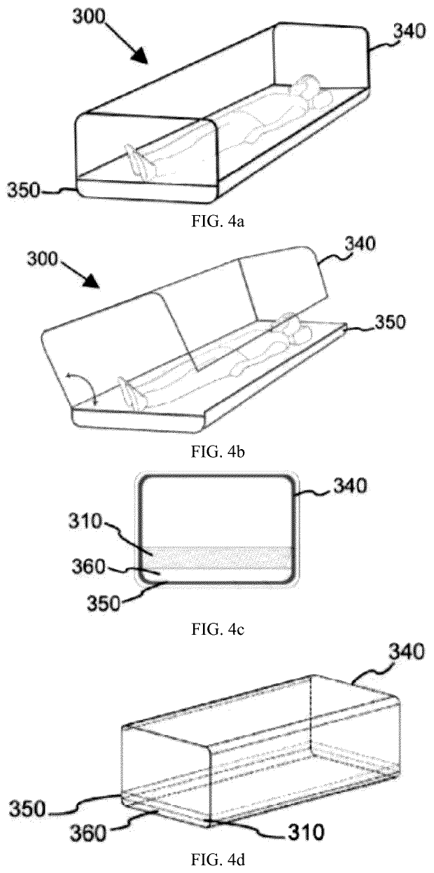

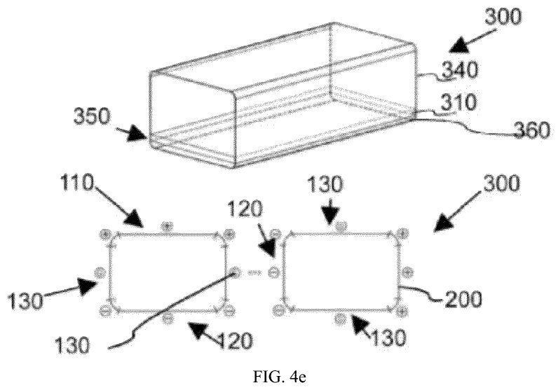

In FIGS. 4a, 4b, 4c and 4d, a representative view of an embodiment where the present invention is integrated to a bed, which is in chamber form, is given.

In FIG. 4e, a representative view of the placement of the grounding electrodes, the first capacitive electrode and the second capacitive electrode in the embodiment where the present invention is integrated to a bed, which is in chamber form, is given.

### REFERENCE NUMBERS

100 Cancer treatment device
110 First capacitive electrode
120 Second capacitive electrode
130 Grounding electrode
140 Signal generator
200 Cancer treatment clothing
201 Front wall
202 Rear wall
203 Side wall
210 Upper body clothing
220 Lower body clothing
230 Headwear
300 Treatment bed
310 Bed layer
320 Base electrode
330 Coverlet
331 Lateral part
332 Central part
340 Cover
350 Base part
360 Dielectrophoresis gap
400 Target region
410 Cancer cell

### DETAILED DESCRIPTION OF THE EMBODIMENTS

In this detailed description, the subject matter is explained with references to examples without forming any restrictive effect only in order to make the subject more understandable.

The cancer cells mentioned in this specification describes benign tumors or malignant tumors.

With reference to FIG. 1, the present invention is a cancer treatment device (100) for treating cancer cells (410) and for preventing spread of cancer cells (410) which exist at a target region (400). The present invention comprises at least one first capacitive electrode (110) and a second capacitive electrode (120) for being positioned at said target region (400). The first capacitive electrode (110) and the second capacitive electrode (120) are positioned such that the target region (400) is in between. A signal generator (140) is provided. The positive end of the signal generator (140) is connected to one of the capacitive electrodes and the negative end of the signal generator (140) is connected to the other one of the capacitive electrodes. The signal generator (140) generates DC signals in waveform in a manner forming electrical field between the first capacitive electrode (110) and the second capacitive electrode (120), in other words, in a manner providing passage of electrical field through the target region (400). Thus, reproduction of the cancel cells (410) is prevented. The signal generator (140) can be DC impact oscillator.

As the basic principle, capacitive electrodes are charged positively and negatively and thereby electrodes are charged and discharged. The charge and discharge process leads to polarization on the outer face of the target region. This electrical polarization generates the electrical waves which enter the target region (400) and which prevent reproduction of the cancer cells (410). In order for this effect to occur, the dielectric coefficient of the medium between the surface and the electrodes must be different from target region (400). In FIG. 1, the polarization which occurs on the outer face of the target region (400) and the electrical waves which occur as a result of this polarization and which pass through the cancer cell (410) are shown.

Here, the mentioned target region (400) can be the body of a patient or a part of the body.

In a possible embodiment of the present invention, grounding electrodes are provided which are positioned in a parallel manner to the electrical field formed by the capacitive electrodes. The grounding electrodes are placed in the vicinity of the target region (400). As can be seen in FIG. 1, the first capacitive electrodes (110) and the second capacitive electrodes (120) are placed in a parallel manner to each other and they take the target region (400) in between. The grounding electrodes are placed mutually in a manner making an angle of 90 degrees with respect to the capacitive electrodes and in a parallel manner to each other and in a parallel manner to the electrical field generated by the capacitive electrodes. The grounding electrodes and capacitive electrodes encircle the target region (400). Thanks to the grounding electrodes, the electrical field is intensified and passes by making reduced amount of electric fringing through the target region (400).

The grounding electrodes are not electrically connected to the capacitive electrodes.

In a possible embodiment of the present invention, the signal generator (140) generates square wave with 50% duty cycle. In more details, the signal generator (140) generates signal at repetition frequencies formed between 50 to 150 kHz and generates signals which change between 100 kHz and 3 MHz. The changes in the signal frequency can be controlled by a processor (not shown in the figures).

In a possible embodiment of the present invention, capacitive electrodes are positioned such that there is at least 1 mm air gap between said capacitive electrodes and the target region (400) outer surface. This air gap is defined as a dielectrophoresis gap (360). Thanks to this, electrical field gradient occurs in the dielectrophoresis gap (360) and the dielectric constant difference between the capacitive electrodes and the target region (400) surface increases the electrical field force (dielectrophoresis) applied to the target region (400). Moreover, dielectric coefficient difference is increased.

With reference to FIG. 2a, in a possible embodiment of the present invention, the cancer treatment device (100) is provided in an embedded manner to cancer treatment clothing (200).

In a possible embodiment of the present invention and with reference to FIG. 2a-2b, the cancer treatment clothing (200) can be upper body clothing (210). The upper body clothing (210) comprises a front wall (201) provided in a manner corresponding to the front face of the upper body of the person who wears said clothing, a rear wall (202) provided in a manner corresponding to the rear face, and side walls (203) which join the front wall (201) and the rear wall (202). With reference to FIG. 2a, the first capacitive electrode (110) and the second capacitive electrode (120) can be provided on said front wall (201) and on said rear wall (202); and the grounding electrodes (130) can be provided on the side walls (203). The upper body clothing (210) is dimensioned in a manner positioned at least 1 mm away from the body of the patient. Thus, dielectrophoresis gap (360) can be provided between the patient's body and the electrodes. In a possible embodiment of the present invention, the dielectrophoresis gap (360) can be provided by means of a fabric having suitable dielectric coefficient and having thickness of 1 mm.

The upper body clothing can be provided in vest form.

In a possible embodiment of the present invention and with reference to FIG. 2d, the cancer treatment clothing (200) can be a lower body clothing (220). The lower body clothing (220) comprises a front wall (201) provided in a manner corresponding to the front face (groin region) of the lower body of the person who wears said clothing, a rear wall (202) provided in a manner corresponding to the rear face, and side walls (203) which join the front wall (201) and the rear wall (202). With reference to FIG. 2d, the first capacitive electrode (110) and the second capacitive electrode (120) can be provided on said front wall (201) and on said rear wall (202); and the grounding electrodes (130) can be provided on the side walls (203). Lower body clothing (220) is dimensioned in a manner positioned at least 1 mm away from the body of the patient. Thus, the dielectrophoresis gap (360) can be provided between the body of the patient and the electrodes. In a possible embodiment of the present invention, the dielectrophoresis gap (360) can be provided by means of a fabric having suitable dielectric coefficient and having thickness of 1 mm. The lower body clothing (220) can be in short form.

In a possible embodiment of the present invention and with reference to FIG. 2c, the cancer treatment clothing (200) can be a headwear (230). The headwear (230) can have a structure which completely encircles the head except the nose and the eye. Capacitive electrodes can be provided on the two mutual side regions at the sides of the head in a manner covering the ears, and grounding electrodes (130) can be provided at the parts which encircle the front, peak region, jaws and back regions of the head. The headwear (230) is dimensioned in a manner positioned at least 1 mm away from the body of the patient. Thus, the dielectrophoresis gap (360) can be provided between the body of the patient and the electrodes. In a possible embodiment of the present invention, the dielectrophoresis gap (360) can be provided by means of a fabric having suitable dielectric coefficient and having thickness of 1 mm. The lower body clothing (220) can be in short form.

Thanks to the distances of at least 1 mm, the fringing effect at the ends of the body is reduced, the electrical field which enters the body is uniformed, the electrical field vortexes are reduced and the electrical field force is increased. In a possible embodiment of the present invention, the dielectrophoresis distances are 1 cm. With the distances of 1 cm, it has been detected that the treatment is the most efficient.

In a possible embodiment of the present invention, the cancer treatment device (100) can be provided in an integrated form to a treatment bed (300). With reference to FIG. 3a-3b, the treatment bed (300) comprises a bed layer (310) for lying of the patient, a base electrode (320) provided under said bed layer (310). One of the first capacitive electrode (110) and the second capacitive electrode (120) has been integrated to the base electrode (320). A coverlet (330) has been provided for covering the patient. Said coverlet (330) is in rectangular form, and a central part (332), which extends in the direction of the height of the patient, comprises lateral parts (331) which remain at the edges of the middle part and which extend in the direction of the height of the patient. The other one of the first capacitive electrode (110) and the second capacitive electrode (120) is provided at the central part (332). Grounding electrodes are provided at the lateral parts (331).

In a possible embodiment of the present invention, the headwear (230) and the treatment bed (300) can be used together. An insulating pillow can be provided for resting of the head of the patient.

In a possible embodiment of the present invention as in FIG. 4a-4b, the cancer treatment device (100) has been integrated to a treatment bed (300) and the cancer treatment bed (300) is provided in a chamber form. In more details, it comprises a conductive base part. The base part is provided in a manner encircling a volume and such that an upper face thereof is open. A bed layer (310) is provided for lying of the patient in the base part. A dielectrophoresis gap (360) is provided between the base of the base part and the bed. A cover (340) is provided for covering the patient. The cover (340) can be electrically connected to the base part (350) such that the cover (340) and base part (350) together form a tubular chamber structure. The cover (340) can be opened and closed on base part (350). Grounding electrodes (130) are provided on the side walls (203) which connect the walls where the capacitive electrodes are provided.

In a possible embodiment of the present invention, the image of the cancer is obtained from PET, PET-MR-, PET-CT, MRI or CT DICOM, etc. images. The characteristic of the electrical field, which will be applied, can be determined beforehand by utilizing this image. Said characteristics can be the electrical field force, and electrical field intensity distribution.

The protection scope of the present invention is set forth in the annexed claims and cannot be restricted to the illustrative disclosures given above, under the detailed description. It is because a person skilled in the relevant art can obviously produce similar embodiments under the light of the foregoing disclosures, without departing from the main principles of the present invention.

What is claimed is:

1. A cancer treatment device for preventing spread of cancer cells existing at a target region, wherein the cancer treatment device comprises at least one first capacitive electrode for being positioned in the vicinity of the target region; a second capacitive electrode for being positioned against the first capacitive electrode such that the target region is positioned in between, and a signal generator for applying wave-formed DC signal to the first capacitive electrode and to the second capacitive electrode forming a variable electrical field between the first capacitive electrode and the second capacitive electrode.

2. The cancer treatment device according to claim 1, wherein the first capacitive electrode and the second capacitive electrode are positioned to form a dielectrophoresis gap equal to a predetermined distance between the outer face of the target region and the first capacitive electrode and the second capacitive electrode.

3. The cancer treatment device according to claim 2, wherein the predetermined distance is at least 1 mm.

4. The cancer treatment device according to claim 1, wherein the signal generator is configured to generate a signal which has square waveform.

5. The cancer treatment device according to claim 4, wherein the signal generator is configured to generate the signal with 50% duty cycle.

6. The cancer treatment device according to claim 5, wherein the signal generator is configured to generate the signal with frequencies which change between 100 kHz and 3 MHz.

7. The cancer treatment device according to claim 1, wherein the cancer treatment device comprises at least one grounding electrode associated with the grounding terminal and provided in the vicinity of the target region in a manner extending substantially parallel to the electrical field formed by the first capacitive electrode and the second capacitive electrode.

8. The cancer treatment device according to claim 7, wherein two grounding electrodes are provided and are positioned mutually on two sides of the target region.

9. A cancer treatment clothing for treating and preventing the recurrence and spread of the cancer cells which exist at a target region comprising the cancer treatment device according to claim 1.

10. The cancer treatment clothing according to claim 9, wherein the cancer treatment clothing is an upper body clothing or a lower body clothing.

11. (canceled)

12. The cancer treatment clothing according to claim 9, wherein the cancer treatment clothing is a headwear.

13. (canceled)

14. The cancer treatment clothing according to claim 10, wherein the cancer treatment clothing comprises a front wall, a rear wall positioned against the front wall, and side walls positioned mutually and which bind the rear wall and the front wall to each other; the first capacitive electrode is provided on the front wall, the second capacitive electrode is provided on the rear wall.

15. The cancer treatment clothing according to claim 13, wherein each grounding electrodes is provided on a side wall of the vests.

16. The cancer treatment clothing according to claim 10, wherein the cancer treatment clothing is a short.

17. The cancer treatment clothing according to claim 9, wherein in order to define a dielectrophoresis gap, the cancer treatment clothing is provided in abundant dimension such that there is distance of at least 1 mm as from the body of the user.

18. A cancer treatment bed for treating and preventing the recurrence and spread of the cancer cells which exist at a target region comprising the cancer treatment device according to claim 1.

19. The cancer treatment bed according to claim 18, wherein the at least one first capacitive electrode is connected to at least one base electrode; a bed layer for lying a patient and providing a dielectrophoresis gap between the base electrode and the patient; a coverlet for covering the patient; and a middle part provided longitudinally in the middle of the coverlet is connected to the second capacitive electrode.

20. The cancer treatment bed according to claim 19, wherein the grounding electrode is provided at the edge parts on the two edges of the middle part.

21. The cancer treatment bed according to claim 18, wherein the cancer treatment bed comprises: a hollow base part having an open upper wall; a bed layer providing a dielectrophoresis gap between the base of the base part and the bed layer; and a cover covering the bed layer and a patient laying on the bed layer.

22. A method for treating and preventing the recurrence and spread of cancer cells at a target region using the cancer treatment device according to claim 1 comprising a step of passing a variable electrical field through the target region, wherein the variable electrical field is formed by the signal generator; the first capacitive electrode is positioned to form a dielectrophoresis gap equal to a distance of at least 1 mm between the target region and the first capacitive electrode; and the second capacitive electrode is positioned to form a dielectrophoresis gap equal to a distance of at least 1 mm between the target region and the second capacitive electrode.
